(19) United States
(12) Patent Application Publication
(10) Pub. No.: US 2010/0274058 A1
(43) Pub. Date: Oct. 28, 2010

(54) Process for Preparation of 2-[Vinyl (Hetero) Arylsulphonyl] Ethanol Derivatives

(76) Inventor: Andrew Whiting, Durham (GB)

Correspondence Address: King & Spalding LLP, P.O. Box 889, Belmont, CA, 94002-0889, US

(21) Appl. No.: 12/224,057

(22) PCT Filed: Feb. 12, 2007
(86) PCT No.: PCT/GB2007/000479

§ 371 Date: Jul. 13, 2010

(30) Foreign Application Priority Data
Feb. 17, 2006 (GB) 0603232.0

(51) Int. Cl. C07C 315/02

(52) U.S. Cl. 568/32

(57) ABSTRACT

The present invention relates to a method of making monomers of the formula X═C(H)—Y—S02-CH2-CH(R)—OH which comprises reacting a compound of the formula Q-Y—S02-CH2-CH(R)—OH with a vinyl-containing organometallic reagent comprising a substituted or unsubstituted moiety X═, wherein X═ is a group selected from CH2=, MeO2C(H)C═, MeO2C(Me)C═ and MeC02C═; Y is an optionally substituted aromatic or heteroaromatic diradical; R is a hydrogen or C1-5 alkyl group; and Q is bromo, chloro, iodo, triflate or tosylate. These monomers are of utility in the preparation of cross-linkable resin compositions

The present invention relates to an improved method of making monomers, which monomers are of utility in the preparation of cross-linkable resin compositions.

A combination of environmental pressures and legislation have forced the global coatings industry to seek alternatives to conventional solvent-borne coating systems that are used, for example, as paints and adhesives. The pressures have been particularly strong (and are still gaining pace) in Europe and North America, and have led to growth in the development and use of high-solids and water-borne coating systems that do not release volatile organic compounds (VOCs) into the environment. Current estimates indicate that around 2 million tonnes of VOCs are emitted annually by the coatings industry. The switch to water-borne systems hai brought with it significant technical difficulties, one of which is the need to match the performance characteristics of traditional solvent-borne alternatives. Despite the massive world-wide effort aimed at this problem, there has until very recently been no system developed which addresses the particular criteria for water-borne coatings.

In WO2004/048448 and in D. J. Berrisford et al., (Chem. Commun. 2005, 5904) the concept is described of in situ generation of vinyl sulfonyl moieties which moieties can participate in cross-linking reactions. Prior to formation of the moieties, resin compositions which comprise precursors to the vinyl sulfonyl moieties are storage-stable; the moieties may be generated by, for example, loss of carrier (e.g. by evaporation of water) from the resin which it is desired to cross-link.

An example of a monomer able to provide vinyl sulfonyl moieties after its incorporation into a (co)polymer is 4-hydroxyethylsulfonyl styrene referred to hereinafter as HESS.

In WO2004/048488 and Berrisford et al. (infra) HESS is described as being prepared by:
(i) formation of p-styrene sodium sulfonyl chloride by reaction of p-styrene sodium sulfinate with phosphorous oxychloride;
(ii) reaction of p-styrene sodium sulfonyl chloride with concentrated aqueous solution hydroxide and powered sodium carbonate in the presence of zinc to affordp-styrene sodium sulfinate; and
(iii) reaction of an acidified, cooled, aqueous solution of the p-styrene sodium sulfinate with ethylene oxide diluted in diethyl ether.

Whilst the preparations disclosed in WO2004/048488 and Berrisford et al. (infra) may be used to provide quantities of HESS useful for laboratory-scale experimentation, difficulties, both environmental and chemical, mean that the existing synthetic methodology (summarised immediately above) is not suitable for the provision of multigram (particularly hundreds of gram) quantities of HESS.

In order to remedy this deficiency in the art and the difficulty hitherto in synthesising compounds related conceptually and/or structurally to HESS, of utility in the in situ generation of reactive moieties of polymers derived from such monomers, the present inventors have surprisingly found that a completely different synthetic strategy may be used to afford not only HESS but other such related compounds.

Thus the invention provides a method of making a compound of formula (I):

X═C(H)—Y—SO₂—CH₂—CH(R)—OH (I)

comprising reacting a compound of formula (II):

Q-Y—SO₂—CH₂—CH(R)—OH (II)

with a vinyl-containing organometallic reagent which comprises a substituted or unsubstituted moiety X═, wherein
X═ is a group selected from CH₂═, MeO₂C(H)C═, MeO₂C(Me)C═ and MeO₂C(Me)C═;
Y is an optionally substituted aromatic or heteroaromatic diradical;
R is hydrogen or a C₁₋₅ alkyl group; and
Q is bromo, chloro, iodo, triflate or tosylate.

Various features of the method of the invention will now be set forth. It is to be understood that, unless the context dictates otherwise, each of these features may be combined with one another and such combinations of various specific features are to be considered to be a part of the disclosure, in addition to the various features as individually described.

Firstly, the overall synthetic strategy is now outlined beginning with a brief discussion of the retrosynthetic strategy devised.

The primary retrosynthetic disconnection made by the inventors is of the bond connecting the phenylidene and vinyl portions of HESS. This disconnection is particularly advantageous because it permits, as is discussed hereinbelow, not only the devising of an alternative, and improved, method of making HESS, but also the synthesis of monomers other than HESS, by replacing the vinyl moiety of HESS with other polymerisable moieties. These include, for example, (meth)acryl as well as vinyl acetate.

The starting materials for the method of the invention, compounds of formula (II), may be prepared by a two-step procedure, starting from commercially available “Q”-substituted aryl and heteroaryl thiols in particular halo-substituted aryl and heteroaryl thiols. These thiols may be reacted to attach the desired (optionally substituted) hydroxyethyl moieties by reaction with an appropriate electrophile. The resultant sulfides may then be oxidised to the corresponding sulfones, with any number of oxidising agents known to the skilled person see March's Advanced Organic Chemistry, M. Smith and J. March, 5th Ed., Wiley, Amsterdam 2001. The various monomers provided by the present invention may be used as substrates for in situ generation of cross-linkable moieties.

In addition to all the advantages mentioned above, the synthesis of compounds of formula (I) may be conducted without use of the zinc with attendant high costs and environmental problems associated with its disposal. Moreover, it is not a requirement of this invention that ethylene oxide be used; advantageously the invention allows for it to be avoided, if this is desired.

Now, the various features of the method of the present invention are set forth.

As noted above, the method of the invention provides a synthesis of compounds of formula (I) by reacting compounds of formula (II) with an optionally substituted vinyl-containing organometallic reagent.

It will be understood that the presence of X═ in compounds of formula (I) permits the preparation of E- and Z-isomers. The invention embraces the preparation of such E- and Z-isomers either singly or in combination.

The vinyl-containing moiety is exchanged for Q in the compounds of formula (II) by reaction of a compound of formula (II) with an appropriate organometallic reagent. Preferably the vinyl-containing moiety is vinyl itself (i.e. H₂C═) which upon reaction with the appropriate compounds of formula (II), viz wherein Y=-1,4-phenylidine and R═H, allows HESS to be prepared. In such reactions, both to prepare HESS, and to prepare other compounds of formula (I), Q is preferably bromo or iodo and we find bromo to be particularly convenient.

Where the vinyl-containing polymer is other than vinyl itself the method of the present invention enables the synthesis of corresponding monomers, such as acrylate- and methacrylate-containing monomers. Alternatively, where X═ is AcO(H)C═, other monomers may be prepared which are compatible with vinyl acetate bulk polymers.

In order to prepare the compounds of formula (I), an organometallic reagent comprising X is generated. This reagent is preferably an organopalladium species but, as is known by the skilled person (see E. Negishi et al. in “Metal catalyzed cross-coupling reactions”. P. J. Stang, F. Diederich eds., Wiley-VCH, Weinheim, 1998), organonickel and organoplatinium reagents may also be used. The subsequent discussion focuses upon the use of organopalladium reagents but the invention is not to be considered to be so limited.

The skilled person will be aware of appropriate reactions for introducing the desired vinyl-containing moiety. General methodology relating to the introduction of the vinyl-containing moiety may be found in J. L. Malleron et al. Handbook of Palladium—Catalysed Reactions, Academic Press, San Diego, 1997; H.-U. Blaser et al., Adv. Synth. Cat., 2004, 346, 1583; and V. Farina, Adv. Synth. Cat., 2004, 346, 1533. The references by Blaser et al. and Farina are particularly useful to the skilled person concerned with optimising the efficiency of catalytic reactions, so as to scale up and improve the efficiency of reactions, for example, for industrial applications. The Malleron et al. article is particularly useful regarding the practise cross-couplings in general, particularly Suzuki and Heck couplings. A preferred type of cross-coupling reaction is the palladium-mediated Suzuki-Miyaura or Heck-Mizoroki reactions (J. L. Malleron et al., H.-U. Blaser et al and V. Farina (infra)). A further preferred type of cross-coupling involves the provision of the vinyl-containing reagent as an organoboron, organotin, organosilicon, organomagnesium or organozinc reagent (see “Metal catalyzed cross-coupling reactions”, P. J. Stang, F. Diederich eds., Wiley-VCH, Weinheim, 1998). The use of vinylboronate when preparing HESS is typical of such a reaction. Preferred examples of particularly convenient reactions are discussed below.

Appropriate substrates of formula (II), preferably wherein Q is bromo, may be reacted with the appropriate vinyl boronate ester (referred to hereinafter as method A), e.g. hexylyene glycol or pinacol vinylboronate ester, according to the protocols of Whiting et al. (A. P. Lightfoot, G. Maw, C. Thirsk, S. Twiddle and A. Whiting, Tetrahedron Lett., 2003, 44, 764; A. P. Lightfoot, S. J. R. Twiddle and A. Whiting, Synlett., 2005, 529; A. R. Hunt, S. K. Stewart and A. Whiting, Tetrahedron Lett., 1993, 34. 3599; S. K. Stewart and A. Whiting, J. Organomet. Chem., 1994, 482, 293). The pinacol vinylboronate is commercially available from Aldrich. The hexylyene ester is used may be prepared according to A. P. Lightfoot, G. Maw, C. Thirsk, S. Twiddle and A. Whiting, Tetrahedron Lett., 2003, 44, 764. Other vinylboronate esters may be prepared according to procedure described in the references provided herein.

Another method (method B) involves reaction of vinylboronic anhydride or vinylboroxine trimer-pyridine complex commercially which are commercially available from Aldrich.

Another method (method C) involves application of the methodology of Molander et al. (J. Org. Chem., 2005, volume 70, 3950) in which so-called vinyl boronate “coupling partners”, such as vinyl trifluoroboronates (e.g. potassium vinyl trifluoroboronate, available from Aldrich), may be employed.

Another method (method D) involves the use of inexpensive vinylsiloxane reagents, such as those reported by S. E. Denmark and C. R. Butler (Org. Lett., 2006, 8, 63).

In methods A to D, above the general procedure (which may be subject to notification by the skilled person as appropriate) involves introduction into dried glassware of an appropriate palladium (II) catalyst (we find palladium (II) acetate to be convenient although other palladium (II) salts may be used), ligand (e.g. triphenyl phosphine) and solvent (e.g. acetonitrile or tetrahydrofuran (THF)). After stirring, further THF, then water, alkali (e.g. sodium hydroxide), a radical quencher (e.g. di-tert-butyl phenol), vinyl-containing species (see methods A-D above) and substrate (i.e. compound of formula (II) above) may be introduced. The resultant mixture is preferably degassed (by any convenient route—e.g sparging with an inert gas such as nitrogen or argon or by the “freeze-pump-thaw” method) prior to heating under inert gas at a temperature of 40-80° C., preferable about 70° C. After heating, preferably for between 30 minutes and 48 hours, and cooling, the mixture may be diluted, palladium species removed (e.g. by filtration over celite) and worked up.

We particularly prefer that a radical inhibitor, such as di-tert-butyl phenol, is present since this engenders stability to the compounds of formula (I). Minute quantities (e.g. 0.001-0.5 wt % preferably 0.01-0.1 wt % relative to the weight of the reactant of formula (II) are convenient. Other radical inhibitors may be used in similar quantities.

As the skilled person will appreciate, the quantities and nature of catalyst, ligand and solvent may be varied depending, amongst other factors, upon the scale and desired rate of the reaction concerned and these may be adjusted as appropriate. For example, specific ligands, as reported by Zapf et al. (Chem. Commun. 2004, 38) may be used in order to promote high catalyst turnover and so save on catalyst costs. Alternatively ligandless palladium at very low loadings according to the methods of M. Leysen and K. Kohler (Synlett, 2005, 1761) may be employed.

Where X═ is, preferably, other than H₂C═ still further palladium-mediated reactions may be practised. For example, a Heck-Mizoroki coupling (A. de Meijere and F. E. Meyer, Angew. Chem. Int. Edn, 1994, 33, 2379 and I. P. Beletskaya and A. V. Cheprakov, Chem. Rev, 2000, 100, 3009) may be used in order to react substrates of formula (II) with methyl acrylate or methyl methacrylate, or with vinyl acetate. Other palladium-medicated cross-coupling reactions will be evident to the skilled person, as alluded to hereinbefore with reference to the articles by Malleron et al., Blaser et al. and Farina (infra), and the preceding discussions of exemplary reactions is intended to be just that.

The compounds of formula (II) may be conveniently prepared, for example, by oxidation of compounds of the sulfur atom in compounds of formula (III):

Q-Y—S—CH₂—CH(R)—OH (III)

(wherein Q, Y and R are as hereinbefore defined).

The literature is replete with methodologies by which sulfides may be oxidised to sulfones. We find hydrogen peroxide with manganese catalysis, Oxone (Alonso et al., Tet. Lett., 2002, 43, 3459 and Synthesis, 2003, 277) and perborate (A. McKillop and J. A. Tarbin, Tetrahedron, 1987, 43, 1753) to be convenient. We find the inexpensive and safe perborate to be particularly convenient. Other protocols will be apparent to those skilled in the art.

The compounds of formula (III) may be prepared, for example, by reaction of a compound of formula (IV)

Q-Y—SH (IV)

with ethylene oxide or with a compound of formula (V)

LG-CH₂—CH(R)—OH (V)

(wherein Q, Y and R are as hereinbefore defined and LG is a leaving group).

In some embodiments of this invention, therefore, ethylene oxide may thus be avoided although it may, be used if desired. Preferably a compound LG in compounds of formula (V) is any leaving group susceptible to nucleophilic displacement, preferably chloro, bromo or iodo, or triflate).

Appropriate compounds of formulae (IV) and (V) are commercially available or are easily synthesised by the skilled person. Where the synthesis of HESS is desired, for example, appropriate precursors are 4-bromothiophenol (a compound of formula (IV)) and 2-chloroethanol (a compound of formula (V)).

In each the compounds of formulae (I), (II), (III) and (IV), Y is an optionally substituted aromatic or heteroaromatic diradical. Preferably the diradical is phenylidine, i.e. 1,2-, 1,3- or 1,4-phenylidine, preferably 1,4-phenylidine.

However the diradical may be a corresponding diradical (formally) derived from pyrrole, thiophene, furan or pyridine.

The diradicals Y may be mono-, bi- or tricyclic. Preferably, however they are mono or bicyclic, particularly preferably monocyclic.

The diradicals Y may be substituted with one or more substituents selected from the group consisting of C₁₋₅ alkyl. Preferably, only one or two substituents, still more preferably, one, is or are present.

The invention will now be described with reference to the following illustrative, but not limitative examples which follow.

### EXAMPLE

### Preparation of HESS

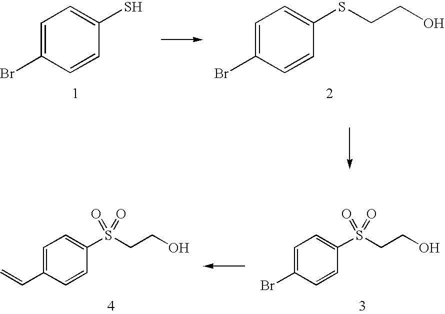

### 1. Alkylation

To a solution/suspension of NaOH (42.5 g, 1.06 mol) and 4-bromothiophenol 1(199.96 g, 1.06 mol) in water (500 mL) was added 2-chloroethanol (71 mL, 1.1 mol) and the mixture stirred at room temperature. After 4 hr the mixture was diluted with brine (100 mL) and DCM (500 mL), separated, re-extracted with DCM (100 mL) and the combined organic phase wahed with brine (200 mL), dried (MgSO₄) and evaporated to give sulfide 2 (224 g, 91%) as a yellow oil. O_{H} (400 MHz, CDCl₃) 2.02 (1H, brs, OH), 3.10 (2H, t, J5.6, CH₂), 3.75 (2H, t, J 5.6, CH₂), 7.25 (211, dt, J8.8 and 2.8, ArH) and 7.42 (2H, dt, J 8.8 and 2.8); S_{c} (100 MHz, CDCl₃) δ7.5 (CH₂), 60.5 (CH₂), 120.7 (ipso ArC), 131.8 (ortho ArC), 132.3 (ortho ArC) and 134.4 (ipso ArC); νₘₐₓ/cm⁻¹ (film) 3480 (broad, OH), 2920 (C—H), 1570 (C═C), 1470 (s), 1290 (s), 1170, 1090 (s), 1060 (s, two bands), 1000 (s) and 804 (arom. C—H bend, 1,4-di subs.); m/z (ES⁺) 232.9631 (MH⁺, C₈H₁₀SOBr⁺ requires 232.9630); m/z (EI) 234, 232, 203, 201, 190, 188, 122 (100%) and 108.

### 2. Oxidation

(i) —H₂O₂ with Mn(acac)₂:

To a stirred solution of 2 (8.30 g, 35.6 mmol) and Mn(acac)₂ (90 mg, 0.36 mmol) in MeCN (140 mL) was added a 0° C. solution of 35% H₂O₂ (50 mL, 0.50 mol) and NaHCO₃ (28 g, 0.33 mol) in water (330 mL) slowly over 90 min. After 30 min the mixture was filtered, the solid was washed with water (50c mL) and EtOAc (100 mL), the phases separated, re-extracted with EtOAc (3×200 mL) and the combined organic phase dried (MgSO₄) and evaporated to give 3 (6.79 g, 72%) as a white solid. δ_{H} (CDCl₃, 500 MHz) 2.62 (1H, t, J 5.5, OH), 3.33-3.37 (2H, m, CH₂OH), 3.99-4.04 (2H, m, CH₂—SO₂), 7.74 (2H, dt, J8.5, 4.5, ArH) and 7.80 (2H, dt, J8.5, 4.5, ArH) Addition of D₂O lead to the disappearance of the peak at 2.62 ppm; S_{c} (CDCl₃, 101 MHz) 56.4 (CH₂OH), 58.5 (CH₂SO₂), 129.6 (Br—C), 129.7 (ortho ArC), 132.9 (ortho ArC) and 138.3 (C—SO₂); Mpt 61.9-63.4° C.; νₘₐₓ/cm⁻¹ (neat) 3480 (O—H), 3090 (C—H), 1640 (C═C), 1570 (C═C), 1470, 1390, 1310 (S═O), 1270 (s, C—O), 1140 (s, S═O) and 840 (arom. C—H bend, 1,4-di subs.); m/z (ES⁴) 281.9794 (M+NH4⁺, C₈H₁₃NO₃SBr⁺ requires 281.9798); m/z (CI⁺) 284 (100%), 282 and 204.

(ii)—Oxone®:

To a solution of 2 (1.00 g, 4.3 mmol) in water/methanol (1:1, 86 mL) was added Oxone® (26.0 g, 43 mmol), portionwise and the mixture stirred at room temperature, after 3 hr more Oxone® (15 g, 24.4 mmol) was added and after 6 hr more Oxone® (10 g, 16 mmol) was added. After 24 hr the mixture was basicified to pH 8 by the addition of sat aq NaHCO₃, filtered, extracted with EtOAc (3×100 mL) and the combined organic washings dried (MgSO₄) and evaporated to give 3 (0.876 g, 77%) as a white solid.

### Boric Acid/H₂O₂:

To a stirred solution of NaOH (0.643 g, 16.1 mmol) and boric acid (0.960 g, 15.5 mmol) in 50% aqueous methanol (40 mL) was added 35% aqueous hydrogen peroxide (1.6 mL, 16 mmol) and the resulting suspension stirred for 2 min prior to the addition of a solution of sulfide 2 (0.995 g, 4.27 mmol) in MeCN (5 mL). The mixture was stirred for 3 hr at 55° C., further 35% aqueous hydrogen peroxide (0.75 mL, 7.5 mmol) added, the mixture stirred at 55° C. for a further 80 min, diluted with water (50 mL) and EtOAc (100 mL), separated, re-extracted with EtOAc (100 mL) and the combined organic phase dried (MgSO₄) and evaporated to give sulfone 3 (1.10 g, 97%) as a white solid.

(iv)—Sodium perborate:

To a solution of sodium perborate tetrahydrate (66 g, 0.428 mol) and sodium hydroxide (2 g, 5 mmol) in water (1 L) was added a solution of sulfide 2 (100 g, 0.428 mol) in MeCN (0.8 L), the stirred mixture heated to 55° C. and 50% H₂O₂ (68 mL, 0.972 mol) added dropwise over 2 hr, the mixture cooled, filtered (solid washed with 20 mL of MTBE), extracted with MTBE (3×300 mL). The organic phase was washed with sat. aq. NaHCO₃ (2×50 mL). The aqueous phases were re-extracted with MTBE (50 mL). The combined organic phase was dried (MgSO₄) and concentrated to give a white syrup which was redissolved in MTBE (250 mL), washed with sat. aq. NaHCO₃ (3×75 mL), dried (MgSO₄) and concentrated to give a white solid 3 (100.6 g, 89%).

### 3. Suzuki Coupling

(i)—Vinyl Boronate:

To a dried Schlenk tube under a positive pressure of argon was added Pd(OAc)₂ (4 mg, 0.018 mmol), PPh₃ (10 mg, 0.038 mmol) and MeCN (0.5 mL). The mixture was stirred for 5 min before the addition of NaOH (120 mg, 3.0 mmol), water (5 mL), MeCN (4.5 mL), 3 (225 mg, 0.85 mmol) and vinylboronate (0.300 mL, 1.7 mmol). The mixture was degassed using the freeze-pump-thaw method (3×) and the stirred mixture heated to 70° C. under argon. After 20 hr the mixture was cooled, diluted with EtOAc (60 mL containing di-tert-butylphenol 0.05 mg/ml) and passed through Celite. The mixture was diluted with water (30 mL), separated, re-extracted with EtOAc (2×30 mL) and the combined organic phase evaporated without drying to give crude product as a viscous yellow oil. ¹H NMR. spectroscopy indicated 4 to be the major species along with a boronate ester. Purification by silica gel chromatography (EtOAc/petroleum ether, 3:7) failed to separate the two species.

(ii)—Vinylboronic Anhydride:

A mixture of Pd(OAc)₂ (20 mg, 0.089 mmol), PPh₃ (40 mg, 0.15 mmol) and THF (1 mL) under argon was stirred for 2 min before the addition of further THF (39 mL), water (4 mL), NaOH (480 mg, 12 mmol), di-tert-butyl phenol (0.2 mg), vinylboronic anhydride (360 mg, 1.5 mmol) and 3 (1.02 g, 3.85 mmol). The mixture was degassed by sparging with argon and heated to 70° C. After 17 hr the mixture was cooled, diluted with EtOAc (100 cm³), passed through Celite, washed with water (30 cm³), and evaporated. The mixture was redissolved in DCM (50 mL), washed with 1% HCl (10 mL) evaporated to give an orange oil (796 mg) which was shown by ¹H NMR spectroscopy to contain 4, DCM (0.5 equiv.) and EtOAc (0.17 equiv.). Accounting for these impurities results in a 75% yield.

(iv)—Potassium vinyltrifluoroborate:

To a stirred mixture of Pd(OAc)₂ (340 mg, 1.51 mmol), PPh₃ (800 mg, 3.05 mmol) and TEM (50 mL) under argon was added NaOH (32.0 g, 0.80 mol), water (100 mL), THF (650 mL), potassium vinyltrifluoroborate (24.1 g, 0.18 mol), di-tert-butyl phenol (50 mg) and sulfone 3 (40.0 g, 0.158 mol), the mixture degassed by sparging with argon and heated to 65° C. with vigorous stirring. The reaction was followed by ¹H NMR spectroscopy, after 6 hr the reaction appeared to have stopped after reaching 50% conversion. Further potassium vinyltrifluoroborate (10.0 g, 74.7 mmol) was added, the mixture stirred for a further 16 hr, cooled, diluted with MTBE (1 L), separated, the aqueous phase diluted with water (200 mL) and extracted with MTBE (500 mL), the combined organic phase passed through Celite, washed with water (200 mL), dried (MgSO₄) and evaporated to give HESS 4 (36.88 g) as a brown oil. Yield of greater than 100% due to impurities. S_{H} (500 MHz, CDCl₃) 3.00 (1H, brs, OH), 3.34 (2H, t, J 5.5, CH₂OH), 3.97 (2H, t, J 5.5, CH₂SO₂), 5.46 (1H, d, J 11.0, CH═CHH), 5.90 (1H, d, J 17.5, CH═CHH), 6.75 (1H, dd, J 17.5, 10.5, CH═CH₂), 7.56 (2H, d, J 8.5, ArH) and 7.86 (2H, d, J 8.0, ArH); δ_{C} (126 MHz, CDCl₃) δ6.4 (CH₂OH), 58.3 (CH₂SO₂), 118.4 (ArCHCH₂), 127.1 (ArCH), 128.4 (ArCH), 135.2 (ArCHCH₂), 137.7 (CCHCH₂) and 143.3 (C—SO₂).

(iv)—Trimethylborate/vinylmagnesium Bromide:

To a stirred solution of anhydrous trimethylborate (5.2 mL, 46.6 mmol) in THF (50 mL) at −78° C. under argon was added vinylmagnesium bromide (50 mL of a 1.0M solution in THF, 50 mmol) dropwise over 45 min, the mixture stirred for 1 hr, warmed to room temperature and quenched by the addition of water (20 mL). Potassium fluoride (2.30 g, 39.6 mmol), NaOH (3.20 g, 80 mmol), THF (50 mL), di-tert-butyl phenol (20 mg) and bromo-sulfone 3 (10.0 g, 37.7 mmol) were added, the mixture degassed by sparging with argon, Pd(OAc)₂ (170 mg, 0.76 mmol) and PPh₃ (400 mg, 1.53 mmol) added and the stirred mixture heated to 70° C. After 3.5 hr the mixture was cooled, diluted with MTBE (250 mL) and water (50 mL), separated, the aqueous phase extracted with MTBE (2×75 mL) and the combined organic phase passed through Celite, washed with water (50 mL), dried (MgSO₄) and evaporated to give an orange oil 4 (9.59 g). ¹H NMR spectroscopy indicated the product to contain 0.35 equivalents THF, accounting for this gives a greater than quantitative yield (105%).

The contents of all patents and other publications are hereby incorporated by reference in their entirety.

1. A method of making a compound of formula (I):
X═C(H)—Y—SO₂—CH₂—CH(R)—OH (I) comprising, reacting a compound of formula (II):
Q-Y—SO₂—CH₂—CH(R)—OH (II)
with a vinyl-containing organometallic reagent which comprises a substituted or unsubstituted moiety X═, wherein
X═ is a group selected from CH₂═, MeO₂C(H)C═, MeO₂C(Me)C═ and MeO₂C(Me)C═;
Y is an optionally substituted aromatic or heteroaromatic diradical;
R is hydrogen or a C₁₋₅ alkyl group; and
Q is bromo, chloro, iodo, triflate or tosylate.

2. The method of claim 1, wherein X═ is H₂C═.

3. The method of claim 1, wherein Y is a diradical formally derived from benzene, pyrrole, thiophene, furan or pyridine.

4. The method of claim 1, wherein Y is substituted with one or more substituents selected from the group consisting of C₁₋₅ alkyl.

5. The method of claim 4, wherein Y is substituted with one or two substituents.

6. The method of claim 4, wherein Y is substituted with one substituent.

7. The method of claim 1, wherein Y is unsubstituted.

8. The method of claim 1, wherein —Y— is 1,4-, 1,3-, or 1,2-phenylidine.

9. The method of claim 1 wherein —Y— is 1,4-phenylidine.

10. The method of claim 1, wherein R is methyl, ethyl or hydrogen.

11. The method of claim 1 where R is hydrogen.

12. The method of claim 1, wherein said method is a method of making 4-hydroxyethylsulfonyl styrene.

13. The method of claim 1, wherein said reacting of the compound of formula (II) with said vinyl-containing organometallic reagent is reacting in a nickel-, palladium-, or platinum-mediated cross-coupling reaction.

14. The method of claim 13, wherein said cross-coupling reaction is palladium-mediated.

15. The method of claim 14, wherein said cross-coupling reaction is a Suzuki-Miyaura or Heck-Mizoroki reaction.

16. The method of claim 13, wherein the vinyl-containing organometallic reagent is an organoboron, organotin, organosilicon, organomagnesium or organozinc reagent.

17. The method of claim 1, wherein Q is bromo or iodo.

18. The method of claim 1, wherein the compound of formula (II) is made by oxidation of a compound of formula (III):
Q-Y—S—CH₂—CH(R)—OH (III)
wherein Q is as defined in claim 1.

19. The method of claim 18, wherein the compound of formula (III) is made by reacting a compound of formula (IV):
Q-Y—SH (IV)
with ethylene oxide or with a compound of the formula (V):
LG-CH₂—CH(R)—OH (V)
wherein Q and Y are as defined in claim 1, and LG is a leaving group.

20. The method of claim 19, wherein the compound of formula (IV) is reacted with a compound of formula (V) wherein LG is selected from chloro, bromo, iodo, triflate and tosylate.

21. The method of claim 19, wherein LG is chloro.
